(19) United States
(12) Patent Application Publication
(10) Pub. No.: US 2010/0310483 A1
(43) Pub. Date: Dec. 9, 2010

(54) Compositions Comprising Phosphoric Acid Ester And Hydrophobically Modified, Crosslinked Anionic Polymers

(75) Inventors: Peter Klug, Grossostheim (DE); Waltraud Simsch, Kelkheim (DE)

Correspondence Address: CLARIANT CORPORATION;INTELLECTUAL PROPERTY DEPARTMENT, 4000 MONROE ROAD, CHARLOTTE, NC, 28205, US

(73) Assignee: CLARIANT INTERNATIONAL LTD., 4132 Muttenz (CH)

(21) Appl. No.: 12/864,384

(22) PCT Filed: Jan. 27, 2009
(86) PCT No.: PCT/EP2009/000499

§ 371 Date: Aug. 24, 2010

(30) Foreign Application Priority Data
Jan. 31, 2008 (DE) 102008006857.8
Jan. 27, 2009 (EP) PCT/EP2009/000499

(51) Int. Cl. A61K 8/92; A61K 31/66; A61K 8/81; A61P 43/00; A61Q 1/14; A61Q 15/00; A61Q 19/00; A61Q 19/10

(52) U.S. Cl. 424/59; 514/143; 424/62; 424/65; 510/136; 510/158; 510/130

(57) ABSTRACT

The invention relates to compositions, comprising the following: I) one or more anionic, cross-linked, hydrophobically modified polymers, wherein the hydrophic modification is carried out by a hydrocarbon group having 6 to 50 carbon atoms, and II) one or more phosphoric acid esters. The compositions are preferably cosmetic, pharmaceutical, or dermatological compositions.

Compositions comprising phosphoric acid ester and hydrophobically modified, crosslinked anionic polymers

The present invention relates to compositions comprising hydrophobically modified, crosslinked, anionic polymers and phosphoric acid esters. The compositions may be, for example, cosmetic, pharmaceutical or dermatological compositions.

Anionic polyelectrolytes are nowadays used widely for thickening cosmetic and pharmaceutical products. Thus, EP-A-0 816 403 and WO 98/00094 describe crosslinked homopolymers of 2-acrylamido-2-methylpropanesulfonates and their use as thickeners. EP-A-0510 246 discloses crosslinked copolymers of N-vinylcarboxamides and unsaturated alkylamides substituted with a sulfonate group, WO 02/43689 describes that hydrophobically modified copolymers based on acrylamidoalkylsulfonates can be used as thickener and stabilizer in cosmetic compositions.

A common feature of all crosslinked polyelectrolytes is that, in an adequate concentration in aqueous systems, they effect the build-up of a yield point which protects suspended solids or else liquid phases against creaming and/or sedimentation. This property is also attained when the anionic, crosslinked polyelectrolyte carries hydrophobic groups. However, disadvantages are the relatively high price of anionic polyelectrolytes and the generally very high salt sensitivity. As soon as the compositions comprise relatively large amounts of salt, the thickening ability of these anionic polyelectrolytes is significantly reduced.

Nonionic associative thickeners such as polyglycol esters (PEG-150 distearate, PEG-150 polyglyceryl-2-tristearate, PEG-120 methylglucose dioleate) or highly ethoxylated phosphoric esters, by contrast, can effect high viscosity on account of their low molecular weight, especially with surfactants, and are salt-insensitive; however, they do not build up a yield point and are therefore not very suitable for stabilizing suspensions or emulsions.

It was therefore an object to provide thickener systems which, for a low use amount, build up a yield point and exhibit a good thickening ability, even in the case of a high salt fraction in the compositions.

Surprisingly, it has been found that this object is achieved by combinations of hydrophobically modified, anionic, crosslinked polyelectrolytes and associative thickeners based on phosphoric acid. These combinations have a higher viscosity ability that the respective individual components and at the same time build up a higher yield point than can be achieved through the anionic polymer alone. Advantageously, using these combinations, it is possible to produce aqueous gels as basis for cosmetic, pharmaceutical and dermatological products. These are characterized by increased salt stability and, on the skin, lose their consistency considerably more slowly and thereby facilitate significantly improved sensorics on the skin.

The invention therefore provides compositions comprising
I) one or more anionic, crosslinked, hydrophobically modified polymers, where the hydrophobic modification takes place through a hydrocarbon radical, preferably an alkyl radical, having 6 to 50, preferably 12 to 40 and particularly preferably 18 to 22 carbon atoms, and
II) one or more phosphoric acid esters

where the one or more phosphoric acid esters comprise:

1) one or more structural units derived from substances of component a), where the substances of component a) are selected from orthophosphoric acid and one or more of their derivatives, and where the one or more derivatives of orthophosphoric acid are preferably selected from polyphosphoric acid, tetraphosphorus decaoxide, phosphorus oxychloride and phosphorus pentachloride,

2) one or more structural units derived from substances of component b), where the substances of component b) are selected from one or more compounds of the formula (I)

R¹—O—(CH₂CH₂O)ᵤ(C₃H₆O)ᵥ(DO)_{w}—H (I)

in which

R¹ is a linear or branched, saturated alkyl group having 6 to 30, preferably 8 to 22, particularly preferably 12 to 18, carbon atoms, is a linear or branched, mono- or polyunsaturated alkenyl group having 6 to 30, preferably 8 to 22, particularly preferably 12 to 18, carbon atoms or is an aryl group, in particular a phenyl group which may be substituted by 1 to 3 branched alkyl groups, which in each case independently of one another comprise 3 to 18 and preferably 4 to 12 carbon atoms,

D is a linear or branched saturated alkylene group having 4 to 20 carbon atoms, is a linear or branched mono- or polyunsaturated alkenylene group having 4 to 20 carbon atoms or is —CH(aryl)CH₂—, where —CH(aryl)CH₂— is preferably —CH(phenyl)CH₂—,

u is a number from 0 to 200, preferably from 2 to 150, particularly preferably from 5 to 100, especially preferably from 10 to 50,

v is a number from 0 to 100, preferably from 0 to 50, particularly preferably from 0 to 20 and especially preferably 0,

w is a number from 0 to 100, preferably from 0 to 20, particularly preferably from 0 to 10 and especially preferably 0, and

where the groups CH₂CH₂O, C₃H₆O and DO from the compounds of formula (I) may be arranged blockwise or in random distribution, and the sum u+v+w is ≧10, preferably ≧20, particularly preferably ≧25, especially preferably ≧30,

3) optionally one or more structural units derived from substances of component c), where the substances of component c) are selected from one or more diols of the formula (II)

HO—(CH₂CH₂O)ₐ(C₃H₆O)_{b}(DO)_{c}—H (II)

in which

D has the meaning as in formula (I),

a is a number from 0 to 800, preferably from 0 to 250, particularly preferably from 10 to 200 and especially preferably from 20 to 100,

b is a number from 0 to 100, preferably from 0 to 50 and especially preferably 0,

c is a number from 0 to 100, preferably from 0 to 20 and especially preferably 0,

where the sum a+b+c is ≧1, preferably from 5 to 150, and the groups CH₂CH₂O, C₃H₆O and DO from the compounds of the formula (II) may be arranged blockwise or in random distribution, and

4) optionally one or more structural units derived from a polyol having more than 2 OH groups.

A preferred embodiment of the invention is compositions comprising, in component I), one or more anionic, crosslinked, hydrophobically modified polymers obtained by copolymerization of

a) acrylic acid, methacrylic acid or C₁-C₄ alkyl esters of acrylic acid or of methacrylic acid,

b) one or more monomers of the formula (III)

R²—Y—(R³—O)ₓ—R⁴ (III)

in which R² is a vinyl, allyl, acryloyl or methacryloyl radical, R³ is (C₂-C₄)-alkylene, preferably CH₂CH₂, R⁴ is a linear or branched, saturated alkyl group having 6 to 50, preferably 12 to 40 and particularly preferably 18 to 22, carbon atoms, x is an integer from 0 to 500, preferably from 0 to 25, and Y is 0, S or NH, preferably O, and

c) one or more crosslinkers,

d) and optionally one or more further monomers (monomers A1).

Particular preference is given to compositions according to the invention comprising, in component I), one or more anionic, crosslinked, hydrophobically modified polymers obtained by copolymerization of acrylic acid and C₁₀₋₃₀-alkylacrylic acid esters, preferably the copolymers available under the trade names Pemulen® TR1 (acrylates/C₁₀₋₃₀ alkyl acrylate copolymer), Pemulen® TR-2 (acrylates/C₁₀-₃₀ alkyl acrylate copolymer) and Carbopol® ETD 2020 (acrylates/C₁₀₋₃₀ alkyl acrylate copolymer), and copolymers obtained by copolymerization of acrylic acid or methacrylic acid and alkoxylated C₆₋₃₀-alkyl acrylic acid esters or C₆₋₃₀-alkyl methacrylic acid esters, preferably the copolymers available under the trade names Aculyn® 22 (acrylates/steareth-20 methacrylate copolymer), Aculyn® 28 (acrylates/beheneth-25 methacrylate copolymer), Synthalen® W 2000 (abrylates/palmeth-25 acrylate copolymer), and Structure® 3001 (acrylates/ceteth-20 itaconate copolymer).

Compositions according to the invention comprising, in component I), one or more anionic, crosslinked, hydrophobically modified polymers selected from hydrophobically modified polyacrylates, preferably acrylates/C₁₀₋₃₀ alkyl acrylate crosspolymer, are especially preferred.

A further preferred embodiment of the invention is compositions comprising, in component I), one or more anionic, crosslinked, hydrophobically modified polymers selected from hydrophobically modified polysulfonic acids or -sulfonates.

Particular preference is given to compositions according to the invention comprising, in component I), one or more anionic, crosslinked, hydrophobically modified polymers obtained by copolymerization of

a) one or more monomers of the formula (IV)

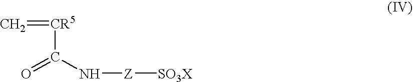

in which R⁵ is hydrogen, methyl or ethyl, Z is (C₁-C₈)-alkylene and X is hydrogen, lithium, sodium, potassium, magnesium, calcium, ammonium, monoalkylammonium, dialkylammonium, trialkylammonium or tetraalkylammonium, where the alkyl substituents of the ammonium ions, independently of one another, are (C₁-C₂₂)-alkyl radicals or (C₂-C₁₀)-hydroxyalkyl radicals,

b) one or more monomers of the formula (V)

R²—Y—(R³—O)ₓ—R⁴ (V)

in which R² is a vinyl, allyl, acryloyl or methacryloyl radical, R³ is (C₂-C₄)-alkylene, preferably CH₂CH₂, R⁴ is a linear or branched, saturated alkyl group having 6 to 50, preferably 12 to 40 and particularly preferably 18 to 22, carbon atoms, x is an integer from 0 to 500, preferably 1 to 50, particularly preferably 6 to 30, and Y is O, S or NH, preferably O, and

c) one or more crosslinkers,

d) and optionally one or more further monomers (monomers A2).

Among the compositions just described, preference is in turn given to those which comprise, in component I), one or more anionic, crosslinked, hydrophobically modified polymers obtained by copolymerization of

a) one or more monomers of the aforementioned formula (IV), in which R⁵ is hydrogen, Z is —C(CH₃)₂—CH₂— and X has the meaning given above under formula (IV),

b) one or more monomers of the aforementioned formula (V), in which R² is methacryloyl, R³ is CH₂CH₂, R⁴ is a radical selected from stearyl, lauryl, cocoyl, undecyl, behenyl, cetearyl, cetyl and myristyl, x is an integer from 3 to 50, preferably 6 to 30, and Y is O, S, or NH, preferably O, and

c) one or more crosslinkers,

d) and optionally one or more further monomers (monomers A3).

Accordingly, particular preference is given to compositions according to the invention comprising, in component I), one or more anionic, crosslinked, hydrophobically modified polymers selected from hydrophobically modified copolymers based on acrylamidomethylpropanesulfonic acids or salts thereof, among these preferably ammonium acryloyldimethyltaurate/steareth-25 crosspolymer (e.g. Aristoflex® HMS, Clariant) or ammonium acryloyldimethyltaurate/beheneth-25 crosspolymer (e.g. Aristoflex® HMB, Clariant).

Preferred monomers A1, A2 and A3 are unsaturated carboxylic acids and anhydrides and salts thereof, and also esters thereof with aliphatic, cycloaliphatic, arylaliphatic or aromatic alcohols with a carbon number of from 1 to 30. Particularly preferred unsaturated carboxylic acids are acrylic acid, methacrylic acid, maleic acid, fumaric acid, crotonic acid, itaconic acid and senecioic acid.

The degree of neutralization of the carboxylic acids can be between 0 and 100%.

Further preferred monomers A1, A2 and A3 are open-chain N-vinylamides, preferably N-vinylformamide (VIFA), N-vinylmethylformamide, N-vinylmethylacetamide (VIMA) and N-vinylacetamide; cyclic N-vinylamides (N-vinyllactams) with a ring size of from 3 to 9, preferably N-vinylpyrrolidone (NVP) and N-vinylcaprolactam; amides of acrylic acid and methacrylic acid, preferably acrylamide, methacrylamide, N,N-dimethylacrylamide, N,N-diethylacrylamide and N,N-diisopropylacrylamide; alkoxylated acryl- and methacrylamides, preferably hydroxymethylmethacrylamide, hydroxyethylmethacrylamide, hydroxypropylmethacrylamide; mono-[2-(methacryloyloxy)ethyl]succinate, N,N-dimethylamino methacrylate, diethylaminomethyl methacrylate, acryl- and methacrylamidoglycolic acid, 2- and 4-vinylpyridine, vinyl acetate, glycidyl methacrylate, styrene, acrylonitrile, vinyl chloride, stearyl acrylate, lauryl methacrylate, vinylidene chloride, tetrafluoroethylene, organic acids, salts and esters thereof, where preferred acids are vinylphosphonic acid, vinylsulfonic acid, allylphosphonic acid, styrenesulfonic acid and methallylsulfonic acid.

Preferred counterions of the salts of the monomers A1, A2 and A3, for example of the salts of the unsaturated carboxylic acids, are Li⁺, Na⁺, K⁺, Mg⁺⁺, Ca⁺⁺, Al⁺⁺⁺, NH₄⁺, quaternary ammonium ions [HNR¹R²R³]⁺, where R¹, R² and R³, independently of one another, are identical or different and may be hydrogen, a linear or branched alkyl group having 1 to 22 carbon atoms, a linear or branched, mono- or polyunsaturated alkenyl group having 2 to 22 carbon atoms, a linear or branched mono-hydroxyalkyl group having 2 to 10 carbon atoms, preferably a mono-hydroxyethyl group or a linear or branched mono-hydroxypropyl group, a linear or branched di-hydroxyalkyl group having 3 to 10 carbon atoms, or (EO)ₙ(PO)ₘH, in which EO is ethyleneoxy, PO is propyleneoxy, n is 0 to 30 and m is 0 to 30 and n+m is ≧2.

The ammonium counterions of the salts of the monomers A1, A2 and A3 may thus be, for example, monoalkylammonium, dialkylammonium or trialkylammonium counterions, where the alkyl groups, independently of one another, are, for example, (C₁-C₂₂)-alkyl radicals or (C₂-C₁₀)-hydroxyalkyl radicals, or the ammonium counterion may also be, for example, mono- to triethoxylated ammonium compounds with different degree of ethoxylation.

In a preferred embodiment of the invention, the monomers A1, A2 and A3 are compounds with a discrete molecular weight.

In a further preferred embodiment of the invention, the one or more anionic, crosslinked, hydrophobically modified polymers comprise no structural units derived from the monomers A1, A2 and A3. The corresponding polymers are in each case obtained by copolymerization of the components a), b) and c).

Suitable crosslinkers are all monomers with at least two olefinic double bonds. Preference is given to methylenebisacrylamide; methylenebismethacrylamide; esters of unsaturated mono- and polycarboxylic acids with polyols, preferably diacrylates and triacrylates or -methacrylates, particularly preferably butanediol and ethylene glycol diacrylate or -methacrylate, trimethylolpropane triacrylate (TMPTA) and trimethylolpropane trimethacrylate (TMPTMA); allyl compounds, preferably allyl (meth)acrylate, triallyl cyanurate, diallyl maleate, polyallyl ester, tetraallyloxyethane, triallylamine, tetraallylethylenediamine; allyl ester of phosphoric acid; and/or vinylphosphonic acid derivatives. Particular preference is given to the crosslinking structures derived from trimethylolpropane triacrylate (TMPTA).

The anionic, crosslinked, hydrophobically modified polymers of the component I) comprise preferably 0.01 to 10% by weight of structural units derived from crosslinking monomers.

The one or more structural units of the component II) 1) the phosphoric acid esters are preferably derived from orthophosphoric acid. These preferred structural units correspond to the formula

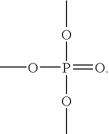

The structural units present in the phosphoric acid esters of component II) of the compositions according to the invention which are derived from substances of component b) selected from the compounds of formula (I) correspond to the formula (I′)

R¹—O—(CH₂CH₂O)ᵤ(C₃H₆O)ᵥ(DO)_{w}- (I′)

in which R¹, D, u, v and w have the meanings given above under formula (I).

The structural units optionally present in the phosphoric acid esters of component II) of the compositions according to the invention which are derived from substances of component c) selected from the compounds of formula (II) correspond to the formula (II′)

—O—(CH₂CH₂O)ₐ(C₃H₆O)_{b}(DO)_{c}- (II′)

in which a, b, c and D have the meanings given above under formula (II).

The phosphoric acid esters of component II) present in the compositions according to the invention comprise no oxygen-oxygen bond —O—O—. The structural units derived from the substances of components a), b), optionally c) and optionally the structural units derived from the one or more polyols having more than 2 OH groups are bonded to one another via only one oxygen atom —O—.

R¹ in the structural units of component II) 2) the phosphoric acid esters derived from the compounds of formula (I) is preferably a linear or branched, saturated alkyl group having 6 to 30, preferably 8 to 22, particularly preferably 12 to 18, carbon atoms, or is a linear or branched, mono- or polyunsaturated alkenyl group having 6 to 30, preferably 8 to 22, particularly preferably 12 to 18, carbon atoms.

A further preferred embodiment of the invention is compositions comprising, in component II), one or more phosphoric acid triesters of the formula (III)

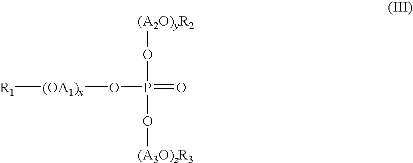

in which

R₁, R₂ and R₃ may be identical or different and are a linear or branched, saturated alkyl group having 6 to 30, preferably 8 to 22 and particularly preferably 12 to 18, carbon atoms, are a linear or branched, mono- or polyunsaturated alkenyl group having 6 to 30, preferably 8 to 22 and particularly preferably 12 to 18, carbon atoms or are an aryl group, in particular a phenyl group, which may be substituted by 1 to 3 branched alkyl groups, which, in each case independently of one another, comprise 3 to 18 and preferably 4 to 12, carbon atoms,

the individual groups (OA₁)ₓ, (A₂O)_{y} and (A₃O)_{z}, in each case independently of one another, consist of units selected from CH₂CH₂O, C₃H₆O and C₄H₃O and where the units CH₂CH₂O, C₃H₆O and C₄H₈O may be arranged within the individual groups (OA₁)ₓ, (A₂O)_{y} and (A₃O)_{z} blockwise or in random distribution, and

x, y and z, in each case independently of one another, are a number from 10 to 150, preferably from 25 to 120, particularly preferably from 40 to 120 and especially preferably from 51 to 100.

In the phosphoric acid triesters of formula (III) just described, R¹, R² and R³ may be identical or different and are preferably linear or branched, saturated alkyl groups having 6 to 30, preferably 8 to 22, particularly preferably 12 to 18, carbon atoms, or are linear or branched, mono- or polyunsaturated alkenyl groups having 6 to 30, preferably 8 to 22, particularly preferably 12 to 18, carbon atoms.

A particularly preferred embodiment of the invention is compositions comprising, in component II), one or more phosphoric acid triesters which have been obtained by reacting phosphoric acid or a phosphoric acid derivative, preferably phosphoric acid, with a fatty alcohol ethoxylate, preferably a fatty alcohol ethoxylate having 10 to 150 EO units (EO═CH₂CH₂O), particularly preferably having 10 to 120 EO units and especially preferably having 20 to 100 EO units and where the fatty alcohol radicals are derived from alcohols selected from octanol, decanol, dodecanol, tetradecanol, hexadecanol, octadecanol, eicosanol, behenyl alcohol, fatty alcohols with C chain cuts between 8 and 22, preferably C₁₀/C₁₂-fatty alcohol, C₁₂/C₁₄-fatty alcohol, C₁₂/C₁₅-fatty alcohol and C₁₆/C₁₈-fatty alcohol, branched fatty alcohols, preferably Guerbet alcohols and monounsaturated fatty alcohols, preferably delta-9-cis-hexadecanol, delta-9-cis-octadecanol, trans-9-octadecanol and cis-delta-11-octadecanol.

An especially preferred embodiment of the invention is compositions comprising, in component II), one or more phosphoric acid triesters which have been obtained by reacting phosphoric acid or a phosphoric acid derivative, preferably phosphoric acid, with C_{16/18}-fatty alcohol ethoxylates having 10 to 150 ethylene oxide units, preferably having 25 to 120 ethylene oxide units, particularly preferably C_{16/18}-fatty alcohol ethoxylate having 25 ethylene oxide units, C_{16/18}-fatty alcohol ethoxylate having 50 ethylene oxide units or C_{16/18}-fatty alcohol ethoxylate having 80 ethylene oxide units.

A further preferred embodiment of the invention is compositions according to the invention comprising, in component II), one or more phosphoric acid esters which comprise structural units derived from compounds of formula (I) and additionally comprise structural units derived from diols of formula (II).

Of these, preference is given to compositions according to the invention comprising, in component II), one or more phosphoric acid esters which comprise structural units derived from diols selected from ethylene glycol, diethylene glycol, triethylene glycol, tetraethylene glycol, polyethylene glycol (PEG) with molecular weights of from 200 to 35 000, preferably PEG 200, PEG 300, PEG 400, PEG 600, PEG 800, PEG 1000, PEG 1500, PEG 2000, PEG 3000, PEG 3350, PEG 4000, PEG 6000, PEG 8000, propylene glycol, dipropylene glycol, tripropylene glycol, polypropylene glycol, polybutylene glycol, copolymers of ethylene oxide and propylene oxide with molecular weights of from 200 to 35 000, 1,2-butanediol, 1,3-butanediol, 1,4-butanediol, 1,2-pentanediol, 1,3-pentanediol, 1,4-pentanediol, 1,5-pentanediol, 1,2-hexanediol, 1,3-hexanediol, 1,4-hexanediol, 1,5-hexanediol, 1,6-hexanediol and 1,12-dodecanediol.

The optionally one or more structural units derived from a polyol having more than 2 OH groups of component II) 4) the phosphoric acid esters are preferably derived from glycerol, diglycerol, polyglycerol, pentaerythritol, dipentaerythritol, pentaerythritol oligomers, trimethylolpropane, threitol, erythritol, adonitol, arabitol, xylitol, mannitol, sorbitol, inositol, glucose, mannose, fructose, sorbose, arabinose, xylose, ribose, mannopyranose, galactopyranose, glucopyranose, maltose, sucrose, amino sugars, ascorbic acid, glucamides and gluconamides, which can also carry one or more alkoxylate groups and where the alkoxylate groups are in each case composed of one or more units selected from CH₂CH₂O, C₃H₆O and C₄H₈O units, which, in each case, may be arranged within the alkoxylate groups blockwise or in random distribution.

The optionally one or more structural units derived from a polyol having more than 2 OH groups of component II) 4) the phosphoric acid esters are particularly preferably derived from pentaerythritol, glycerol and diglycerol, of these preferably pentaerythritol, which can also carry one or more alkoxylate groups and where the alkoxylate groups are in each case composed of one or more units selected from CH₂CH₂O, C₃H₆O and C₄H₈O units which, in each case, may be arranged within the alkoxylate groups blockwise or in random distribution.

In a preferred embodiment of the invention, the just-mentioned preferred and particularly preferred structural units derived from a polyol having more than 2 OH groups of component II) 4) the phosphoric acid esters carry no alkoxylate groups.

In a further preferred embodiment of the invention, the just-mentioned preferred and particularly preferred structural units derived from a polyol having more than 2 OH groups of component II) 4) the phosphoric acid esters carry one or more alkoxylate groups. Among these phosphoric acid esters, preference is given to those in which the alkoxylate groups consist of CH₂CH₂O groups and the number of CH₂CH₂O groups per polyol molecule having more than 2 OH groups is from 1 to 150, preferably from 5 to 130 and particularly preferably from 10 to 110.

Preferably, the one or more phosphoric acid esters of component II) of the compositions according to the invention consist of the structural units II) 1) and II) 2) and optionally II) 3 and II) 4).

In a particularly preferred embodiment of the invention, the phosphoric acid esters of component II) of the compositions according to the invention comprise no structural units derived from a polyol having more than 2 OH groups of component II) 4).

In an especially preferred embodiment of the invention, the phosphoric acid esters of component II) of the compositions according to the invention consist of one or more of the structural units of component II) 1) and one or more structural units of component II) 2).

In a further particularly preferred embodiment of the invention, the phosphoric acid esters of component II) of the compositions according to the invention comprise one or more of the structural units of component II) 1), one or more structural units of component II) 2) and one or more structural units of component II) 3). Among this embodiment, preference is in turn given to the phosphoric acid esters which consist of one or more of the structural units of component II) 1), one or more structural units of component II) 2) and one or more structural units of component II) 3).

The compositions according to the invention comprise the one or more anionic, crosslinked, hydrophobically modified polymers of component I), based on the total weight of the compositions, preferably in an amount of from 0.1 to 3.0% by weight, particularly preferably from 0.2 to 2.0% by weight and especially preferably from 0.3 to 1.0% by weight.

The compositions according to the invention comprise the one or more phosphoric acid esters of component II), based on the total weight of the compositions, preferably in an amount of from 0.1 to 5.0% by weight, particularly preferably from 0.5 to 3.0% by weight and especially preferably from 0.7 to 2.0% by weight.

The combination according to the invention of phosphoric acid esters and hydrophobically modified, crosslinked, anionic polymers exhibits a synergistically enhanced thickening ability both in aqueous- or aqueous-alcoholic-based compositions and also in aqueous-surfactant-based compositions. Yield points of from 15 to 40 Pa are built up.

The compositions according to the invention comprising phosphoric acid esters and hydrophobically modified, crosslinked, anionic polymers have an advantageous, nonsticky consistency.

In a preferred embodiment of the invention, the compositions are cosmetic, pharmaceutical or dermatological compositions.

In a further preferred embodiment of the invention, the compositions according to the invention, preferably the cosmetic, pharmaceutical or dermatological compositions, are present in the form of gels, preferably in the form of hair gels, moisturizing gels, antiperspirant gels, bleaching gels, anti-aging gels, self-tanning gels, sun protection gels, skin whitening gels, conditioners in gel form or disinfectant gels.

A further preferred embodiment of the invention are aqueous-surfactant-based compositions, preferably cosmetic, pharmaceutical or dermatological compositions, particularly preferably shampoos, shower baths, shower gels or foam baths.

It is particularly advantageous that the thickening ability is pronounced also in a strongly acidic medium and in electrolyte-containing compositions.

The combinations according to the invention are therefore particularly suitable for thickening and stabilizing acidic compositions, preferably acidic cosmetic, pharmaceutical or dermatological compositions. These may be, for example, compositions, preferably cosmetic, pharmaceutical or dermatological compositions, which comprise hydroxy acids, such as lactic acid, glycolic acid, salicylic acid, citric acid or carboxylic acids, such as benzoic acid, sorbic acid or polyglycol dioic acids in free or partial neutralization. Furthermore, compositions comprising vitamin C or vitamin C derivatives, dihydroxyacetone or skin-whitening, actives such as arbutin or glycyrrhetic acid and salts thereof can be stabilized.

In a further preferred embodiment of the invention, the compositions according to the invention have a pH of from 2 to 10, preferably from 2 to 6, particularly preferably from 2.5 to 5 and especially preferably from 3 to 4.5.

The compositions according to the invention are preferably electrolyte-containing compositions.

In a further preferred embodiment of the invention, the compositions according to the invention therefore comprise one or more electrolytes.

The electrolytes used are inorganic salts, preferably ammonium or metal salts, particularly preferably of halides, among them in turn preferably CaCl₂, MgCl₂, LiCl, KCl, NaCl, carbonates, hydrogencarbonates, phosphates, sulfates, nitrates, especially preferably sodium chloride, and/or organic salts, preferably ammonium or metal salts, particularly preferably of glycolic acid, lactic acid, citric acid, tartaric acid, mandelic acid, salicylic acid, ascorbic acid, pyruvic acid, fumaric acid, retinoic acid, sulfonic acids, benzoic acid, kojic acid, fruit acid, malic acid, gluconic acid or galacturonic acid.

As electrolyte, the compositions according to the invention can also comprise mixtures of different salts.

The electrolyte-containing compositions according to the invention also include aqueous antiperspirant formulations comprising aluminum salts, preferably aluminum chlorohydrate or aluminum-zirconium complex salts.

The content of the one or more electrolytes in the,compositions according to the invention is, based on the total composition according to the invention, preferably from 0.1 to 20.0% by weight, particularly preferably from 0.2 to 10.0% by weight and especially preferably from 0.5 to 5.0% by weight.

It is very advantageous that the compositions according to the invention which comprise oxidizing agents, preferably hydrogen peroxide, both thicken and also stabilize, for example, hair colorants.

In a further preferred embodiment of the invention, the compositions according to the invention comprise hydrogen peroxide or hydrogen-peroxide-releasing substances. These compositions are preferably present in the form of gels.

Suitable hydrogen-peroxide-releasing substances are preferably inorganic peracids, preferably peroxosulfuric acid, peroxodisulfuric acid, peroxocarbonates, and organic peracids, preferably peracetic acid.

In a further preferred embodiment of the invention, the compositions according to the invention are acidic hydrogen peroxide bleaching gels or creams.

The compositions according to the invention can comprise anionic, cationic, nonionic, ampholytic surfactants and/or betaine surfactants.

The total amount of the surfactants used in the compositions according to the invention (e.g. in the case of rinse-off products) is, based on the finished compositions according to the invention, preferably 1 to 70% by weight, particularly preferably 5 to 40% by weight and especially preferably 10 to 35% by weight.

The anionic surfactants are preferably (C₁₀-C₂₂)-alkyl and alkylene carboxylates, alkyl ether carboxylates, fatty alcohol sulfates, fatty alcohol ether sulfates, alkylamide sulfates and sulfonates, fatty acid alkylamide polyglycol ether sulfates, alkanesulfonates and hydroxyalkanesulfonates, olefinsulfonates, acyl, esters of isethionates, α-sulfo fatty acid esters, alkylbenzenesulfonates, alkylphenol, glycol ether sulfonates, sulfosuccinates, sulfosuccinic acid half-esters and diesters, fatty alcohol phosphates, fatty alcohol ether phosphates, protein-fatty acid condensation products, alkyl monoglyceride sulfates and sulfonates, alkyl glyceride ether sulfonates, fatty acid methyl taurides, fatty acid sarcosinates, sulforicinoleates, acyl glutamates and acyl glycinates. These compounds and mixtures thereof are used in the form of their water-soluble or water-dispersible salts, for example the sodium, potassium, magnesium, ammonium, mono-, di- and triethanolammonium, and analogous alkylammonium salts.

The amount of anionic surfactants in the compositions according to the invention is preferably from 2 to 30% by weight, particularly preferably from 5 to 25% by weight and especially preferably from 12 to 22% by weight, based on the finished compositions.

Preferred cationic surfactants are quaternary ammonium salts, such as di(C₈-C₂₂)-alkyldimethylammonium chloride or bromide, preferably di(C₈-C₂₂)-alkyldimethylammonium chloride or bromide; (C₆-C₂₂)-alkyldimethylethylammonium chloride or bromide; (C₈-C₂₂)-alkyltrimethylammonium chloride or bromide, preferably cetyitrimethylammonium chloride or bromide and (C₈-C₂₂)-alkyltrimethylammonium chloride or bromide; (C₁₀-C₂₄)-alkyldimethylbenzylammonium chloride or bromide, preferably (C₁₂-C₁₈)-alkyldimethylbenzylammonium chloride, (C₈-C₂₂)-alkyldimethylhydroxyethylammonium chloride, phosphate, sulfate, lactate, (C₈-C₂₂)-alkylamidopropyltrimethylammonium chloride, methosulfate, N,N-bis(2-C₈-C₂₂-alkanoyloxyethyl)dimethylammonium chloride, methosulfate, N,N-bis(2-C₈-C₂₂-alkanoyloxyethyl)hydroxyethylmethylammonium chloride, methosulfate.

The amount of cationic surfactant in the compositions according to the invention is preferably 0.1 to 10% by weight, particularly preferably 0.5 to 7% by weight and especially preferably 1 to 5% by weight, based on the finished compositions.

Preferred nonionic surfactants are fatty alcohol ethoxylates (alkylpolyethylene glycols); alkylphenol polyethylene glycols; fatty amine ethoxylates (alkylaminopolyethylene glycols); fatty acid ethoxylates (acyl polyethylene glycols); polypropylene glycol ethoxylates (Pluronics®); fatty acid alkanolamides, (fatty acid amide polyethylene glycols); sucrose esters; sorbitol esters and sorbitan esters and polyglycol ethers thereof, and also C₈-C₂₂-alkyl polyglucosides.

The amount of nonionic surfactants in the compositions according to the invention (e.g. in the case of rinse-off products) is preferably in the range from 1 to 20% by weight, particularly preferably from 2 to 10% by weight and especially preferably from 3 to 7% by weight, based on the finished compositions.

Furthermore, the compositions according to the invention can comprise amphoteric surfactants. These can be described as derivatives of long-chain secondary or tertiary amines which have an alkyl group with 8 to 18 carbon atoms and in which a further group is substituted by an anionic group which imparts the solubility in water, thus, for example, by a carboxyl, sulfate or sulfonate group. Preferred amphoteric surfactants are N—(C₁₂-C₁₈)-alkyl-β-aminopropionates and N—(C₁₂-C₁₈)-alkyl-β-iminodipropionates as alkali metal and mono-, di- and trialkylammonium salts; suitable further surfactants are also amine oxides. These are oxides of tertiary amines with a long-chain group having 8 to 18 carbon atoms and two mostly short-chain alkyl groups having 1 to 4 carbon atoms. Preference is given here, for example, to the C₁₀- to C₁₈-alkyldimethylamine oxides, fatty acid amidoalkyldimethylamine oxide.

A further preferred group of surfactants is betaine surfactants, also called zwitterionic surfactants. These contain in the same molecule a cationic group, in particular an ammonium group and an anionic group. Which may be a carboxylate group, sulfate group or sulfonate group. Suitable betaines are preferably alkylbetaines such as cocobetarne or fatty acid alkylamidopropylbetaines, for example cocoacylamidopropyldimethylbetaine or the C₁₂- to C₁₅-dimethylaminohexanoates and/or the C₁₀- to C₁₈-acylamidopropanedimetylbetaines.

The amount of amphoteric surfactants and/or betaine surfactants in the compositions according to the invention is preferably from 0.5 to 20% by weight and particularly preferably from 1 to 10% by weight, based on the finished compositions.

Preferred surfactants are lauryl sulfate, laureth sulfate, cocoamidopropylbetaine, alkylbetaines such as cocobetaine, sodium cocoyl glutamate and lauroamphoacetate.

In a further preferred embodiment of the invention, the compositions according to the invention additionally also comprise, as foam-boosting agents, cosurfactants from the group of alkylbetaines, alkylamidobetaines, aminopropionates, aminoglycinates, imidazolinium betaines and sulfobetaines, amine oxides, fatty acid alkanolamides and polyhydroxyamides.

In a preferred embodiment of the invention, the compositions according to the invention, preferably the cosmetic, pharmaceutical or dermatological compositions, comprise one or more surfactants.

The compositions according to the invention can comprise, as further auxiliaries and additives, oil bodies, silicone oils, waxes, emulsifiers, coemulsifiers, solubilizers, stabilizers, cationic polymers, film formers, thickeners, gelling agents, superfatting agents, refitting agents, antimicrobial active ingredients, biogenic active ingredients, astringents, deodorizing agents, sun protection filters, antioxidants, humectants, solvents, dyes, fragrances, pearlizing agents, opacifiers and/or water-soluble silicones.

The oil bodies can advantageously be selected from the group of triglycerides, natural and synthetic fatty substances, preferably esters of fatty acids with alcohols of low carbon number, e.g. with isopropanol, propylene glycol or glycerol, or esters of fatty alcohols with alkanoic acids of low carbon number or with fatty acids or from the group of alkyl benzoates, and also natural or synthetic hydrocarbon oils.

Triglycerides of linear or branched, saturated or unsaturated, optionally hydroxylated, C₈-C₃₀-fatty acids, in particular vegetable oils, such as sunflower oil, corn oil, soybean oil, rice oil, jojoba oil, babussu oil, pumpkin oil, grapeseed oil, sesame oil, walnut oil, apricot oil, orange oil, wheatgerm oil, peach kernel oil, macadamia oil, avocado oil, sweet almond oil, lady's smock oil, castoroil, olive oil, peanut oil, rapeseed oil and coconut oil, and also synthetic triglyceride oils, e.g. the commercial product Myritol® 318, are suitable. Hydrogenated triglycerides are also preferred. Oils of animal origin, for example beef tallow, perhydrosqualene, lanolin, can also be used.

A further class of preferred oil bodies is the benzoic acid esters of linear or branched C₈₋₂₂-alkanols, e.g. the commercial products Finsolv® SB (isostearyl benzoate), Finsolv® TN (C₁₂-C₁₅-alkyl benzoate) and Finsolv® EB (ethylhexyl benzoate).

A further class of preferred oil bodies is the dialkyl ethers having in total 12 to 36 carbon atoms, in particular having 12 to 24 carbon atoms, such as, for example, di-n-octyl ether (Cetiol® OE), di-n-nonyl ether, di-n-decyl ether, di-n-undecyl ether, di-n-dodecyl ether, n-hexyl n-octyl ether, n-octyl n-decyl ether, n-decyl n-undecyl ether, n-undecyl n-dodecyl ether and n-hexyl n-undecyl ether, di-3-ethyldecyl ether, tert-butyl n-octyl ether, isopentyl n-octyl ether and 2-methylpentyl n-octyl ether, and di-tert-butyl ether and diisopentyl ether.

Branched saturated or unsaturated fatty alcohols having 6-30 carbon atoms, e.g. isostearyl alcohol, and Guerbet alcohols, are likewise suitable.

A further class of preferred oil bodies is hydroxycarboxylic acid alkyl esters. Preferred hydroxycarboxylic acid alkyl esters are full esters of glycolic acid, lactic acid, malic acid, tartaric acid or citric acid. Further esters of hydroxycarboxylic acids which are suitable in principle are esters of 3-hydroxypropionic acid, of tartronic acid, of D-gluconic acid, sugar acid, mucic acid or glucuronic acid. Suitable alcohol components of these esters are primary, linear or branched aliphatic alcohols having 8 to 22 carbon atoms. Here, the esters of C₁₂-C₁₅-fatty alcohols are particularly preferred. Esters of this type are commercially available, e.g. under the trade name Cosmacol® from EniChem, Augusta lndustriale.

A further class of preferred oil bodies is dicarboxylic acid esters of linear or branched C₂-C₁₀-alkanols, such as di-n-butyl adipate (Cetiol® B), di-(2-ethylhexyl)adipate and di-(2-ethylhexyl)succinate, and also diol esters, such as ethylene glycol dioleate, ethylene glycol diisotridecanoate, propylene glycol di(2-ethylhexanoate), propylene glycol diisostearate, propylene glycol dipelargonate, butanediol diisostearate and neopentyl glycol dicaprylate, and also diisotridecyl azelate.

Likewise preferred oil bodies are symmetrical, asymmetrical or cyclic esters of carbonic acid with fatty alcohols, glycerol carbonate or dicaprylyl carbonate (Cetiol® CC).

A further class of preferred oil bodies is the esters of dimers of unsaturated C₁₂-C₂₂-fatty acids (dimer fatty acids) with monovalent linear, branched or cyclic C₂-C₁₈-alkanols or with polyvalent linear or branched C₂-C₈-alkanols.

A further class of preferred oil bodies is hydrocarbon oils, for example those with linear or branched, saturated or unsaturated C₇-C₄₀-carbon chains, for example Vaseline, dodecane, isododecane, cholesterol, lanolin, synthetic hydrocarbons such as polyolefins, in particular polyisobutene, hydrogenated polyisobutene, polydecane, and hexadecane, isohexadecane, paraffin oils, isoparaffin oils, e.g. the commercial products of the Permethyl® series, squalane, squalene, and alicyclic hydrocarbons, e.g. the commercial product 1,3-di(2-ethylhexyl)cyclohexane (Cetiol® S), ozokerite, and ceresine.

Silicone oils and silicone waxes which are available are preferably dimethylpolysiloxanes and cyclomethicones, polydialkylsiloxanes R₃SiO(R₂SiO)ₓSiR₃, where R is methyl or ethyl, particularly preferably methyl, and x is a number from 2 to 500, for example the dimethicones available under the trade names VICASIL (General Electric Company), DOW CORNING 200, DOW CORNING 225, DOW CORNING 200 (Dow Corning Corporation), and also the dimethicones available under SilCare® Silicone 41M65, SilCare® Silicone 41M70, SilCare® Silicone 41M80 (Clariant), stearyldimethylpolysiloxane, C₂₀-C₂₄-alkyldimethylpolysiloxane, C₂₄-C₂₈-alkyldimethylpolysiloxane, but also the methicones available under SilCare® Silicone 41M40, SilCare® Silicone 41M50 (Clariant), furthermore trimethylsiloxysilicates [(CH₂)₃SiO)_{1/2}]ₓ[SiO₂]_{y}, where x is a number from 1 to 500 and y is a number from 1 to 500, dimethiconols R₃SiO[R₂SiO]ₓSiR₂OH and HOR₂SiO[R₂SiO]ₓSIR₂OH, where R methyl or ethyl and x is a number up to 500, polyalkylarylsiloxanes, for example the polymethyiphenylsiloxanes available under the trade names SF 1075 METHYLPHENYL FLUID (General Electric Company) and 556 COSMETIC GRADE PHENYL TRIMETHICONE FLUID (Dow Corning Corporation), polydiarylsiloxanes, silicone resins, cyclic silicones and amino-, fatty-acid-, alcohol-polyether-, epoxy-, fluorine- and/or alkyl-modified silicone compounds, and also polyether siloxane copolymers.

The compositions according to the invention can comprise waxes, for example paraffin waxes, microwaxes and ozokerites, beeswax and its part fractions, and also beeswax derivatives, waxes from the group of homopolymeric polyethylenes or copolymers of α-olefins, and natural waxes such as rice wax, candelilla wax, carnauba wax, Japan wax or shellac wax.

Emulsifiers, coemulsifiers and solubilizers which can be used are nonionic, anionic, cationic or amphoteric surface-active compounds.

Suitable nonionogenic surface-active compounds are preferably: addition products of from 0 to 30 mol of ethylene oxide and/or 0 to 5 mol of propylene oxide onto linear fatty alcohols having 8 to 22 carbon atoms, onto fatty acids having 12 to 22 carbon atoms, onto alkylphenols having 8 to 15 carbon atoms in the alkyl group and onto sorbitan or sorbitol esters; (C₁₂-C₁₈)-fatty acid mono- and diesters of addition products of from 0 to 30 mol of ethylene oxide onto glycerol; glycerol mono- and diesters and sorbitan mono- and diesters of saturated and unsaturated fatty acids having 6 to 22 carbon atoms and optionally ethylene oxide addition products thereof; addition products of from 15 to 60 mol of ethylene oxide onto castor oil and/or hydrogenated castor oil; polyol and in particular polyglycerol esters, such as, for example, polyglycerol polyricinoleate and polyglycerol poly-12-hydroxyStearate. Ethoxylated fatty amines, fatty acid amides, fatty acid alkanolamides and mixtures of compounds of two or more of these substance classes are likewise preferably suitable.

Suitable ionogenic coemulsifiers are, for example, anionic emulsifiers, such as mono-, di- or triphosphoric acid esters, soaps (e.g. sodium stearate), fatty alcohol sulfates, but also cationic emulsifiers such as mono-, di- and trialkyl quats and polymeric derivatives thereof.

Suitable amphoteric emulsifiers are preferably alkylaminoalkylcarboxylic acids, betaines, sulfobetaines and imidazoline derivatives.

Fatty alcohol ethoxylates selected from the group of ethoxylated stearyl alcohols, isostearyl alcohols, cetyl alcohols, isocetyl alcohols, oleyl alcohols, lauryl alcohols, isolauryl alcohols, cetylstearyl alcohols, in particular polyethylene glycol(13) stearyl ether, polyethylene glycol(14) stearyl ether, polyethylene glycol(15) stearyl ether, polyethylene glycol(16) stearyl ether, polyethylene glycol(17) stearyl ether, polyethylene glycol(18) stearyl ether, polyethylene glycol(19) stearyl ether, polyethylene glycol(20) stearyl ether, polyethylene glycol(12) isostearyl ether, polyethylene glycol(13) isostearyl ether, polyethylene glycol(14) isostearyl ether, polyethylene glycol(15) isostearyl ether, polyethylene glycol(16) isostearyl ether, polyethylene glycol(17) isostearyl ether, polyethylene glycol(18) isostearyl ether, polyethylene glycol(19) isostearyl ether, polyethylene glycol(20) isostearyl ether, polyethylene glycol(13) cetyl ether, polyethylene glycol(14) cetyl ether, polyethylene glycol(15) cetyl ether, polyethylene glycol(16) cetyl ether, polyethylene glycol(17) cetyl ether, polyethylene glycol(18) cetyl ether, polyethylene glycol(19) cetyl ether, polyethylene glycol(20) cetyl ether, polyethylene glycol(13) isocetyl ether, polyethylene glycol(14) isocetyl ether, polyethylene glycol(15) isocetyl ether, polyethylene glycol(16) isocetyl ether, polyethylene glycol(17) isocetyl ether, polyethylene glycol(18) isocetyl ether, polyethylene glycol(19) isocetyl ether, polyethylene glycol(20) isocetyl ether, polyethylene glycol(12) oleyl ether, polyethylene glycol(13) oleyl ether, polyethylene glycol(14) oleyl ether, polyethylene glycol(15) oleyl ether, polyethylene glycol(12) lauryl ether, polyethylene glycol(12) isolauryl ether, polyethylene glycol(13) cetylstearyl ether, polyethylene glycol(14) cetylstearyl ether, polyethylene glycol(15) cetylstearyl ether, polyethylene glycol(16) cetylstearyl ether, polyethylene glycol(17) cetylstearyl ether, polyethylene glycol(18) cetylstearyl ether, polyethylene glycol(19) cetylstearyl ether are particularly preferably used.

Fatty acid ethoxylates selected from the group of ethoxylated stearates, isostearates and oleates, in particular polyethylene glycol(20) stearate, polyethylene glycol(21) stearate, polyethylene glycol(22) stearate, polyethylene glycol(23) stearate, polyethylene glycol(24) stearate, polyethylene glycol(25) stearate, polyethylene glycol(12) isostearate, polyethylene glycol(13) isostearate, polyethylene, glycol(14) isostearate, polyethylene glycol(15) isostearate, polyethylene glycol(16) isostearate, polyethylene glycol(17) isostearate, polyethylene glycol(18) isostearate, polyethylene glycol(19) isostearate, polyethylene glycol(20) isostearate, polyethylene glycol(21) isostearate, polyethylene glycol(22) isostearate, polyethylene glycol(23) isostearate, polyethylene glycol(24) isostearate, polyethylene glycol(25) isostea rate, polyethylene glycol(12) oleate, polyethylene glycol(13) oleate, polyethylene glycol(14) oleate, polyethylene glycol(15) oleate, polyethylene glycol(16) oleate, polyethylene glycol(17) oleate, polyethylene glycol(18) oleate, polyethylene glycol(19) oleate polyethylene glycol(20)oleate are likewise preferred,

Sodium laureth-11 carboxylate can advantageously be used as ethoxylated alkylether carboxylic acid or salts thereof.

Ethoxylated triglycerides which can be used are advantageously polyethylene glycol(60) evening primrose glycerides.

It is furthermore advantageous to select the polyethylene glycol glycerol fatty acid esters from the group polyethylene glycol(20) glyceryl laurate, polyethylene glycol(6) glyceryl caprate/caprinate, polyethylene glycol(20) glyceryl oleate, polyethylene glycol(20) glyceryl isostearate and polyethylene glycol(18) glyceryl oleate/cocoate.

Among the sorbitan esters, polyethylene glycol(20) sorbitan monolaurate, polyethylene glycol(20) sorbitan monostearate, polyethylene glycol(20) sorbitan monoisostearate, polyethylene glycol(20) sorbitan monopalmitate, polyethylene glycol(20) sorbitan monooleate are particularly suitable.

Particularly advantageous coemulsifiers are glyceryl monostearate, glyceryl monooleate, diglyceryl monostearate, glyceryl isostearate, polyglyceryl-3 oleate, polyglyceryl-3 diisostearate, polyglyceryl-4 isostearate, polyglyceryl-2 dipolyhydroxystearate, polyglyceryl-4 dipolyhydroxystearate, PEG-30 dipolyhydroxystearate, diisostearoyl polyglyceryl-3 diisostearate, glycol distearate and polyglyceryl-3 dipolyhydroxystearate, sorbitan monoisostearate, sorbitan stearate, sorbitan oleate, sucrose distearate, lecithin, PEG-7-hydrogenated castor oil, cetyl alcohol, stearyl alcohol, behenyl alcohol, isobehenyl alcohol and polyethylene glycol(2) stearyl ether (steareth-2), alkylmethicone copolyols and alkyldimethicone copolyols, in particular cetyldimethicone copolyol, laurylmethiconecopolyol.

The compositions according to the invention can comprise one or more of the emulsifiers, coemulsifiers or solubilizers in amounts of from 0.1 to 20% by weight, preferably 1 to 15% by weight and particularly preferably 3 to 10% by weight, based on the finished compositions.

Stabilizers which can be used are metal salts of fatty acids, such as, for example, magnesium stearate, aluminum stearate and/or zinc stearate, preferably in amounts of from 0.1 to 10% by weight, preferably 0.5 to 8% by weight and particularly preferably 1 to 5% by weight, based on the finished compositions.

Suitable cationic polymers are those known under the INCl name “Polyquaternium”, in particular Polyquaternium-31, Polyquaternium-16, Polyquaternium-24, Polyquaternium-7, Polyquaternium-22, Polyquaternium-39, Polyquaternium-28, Polyquaternium-2, Polyquaternium-10, Polyquaternium-11, and Polyquaternium 37&mineral oil&PPG trideceth (Salcare SC95), PVP-dimethylaminoethyl methacrylate copolymer, guar hydroxypropyltriammonium chloride, and calcium alginate and ammonium alginate. Furthermore, cationic cellulose derivatives;, cationic starch; copolymers of diallylammonium salts and acrylamides; quaternized vinylpyrrolidone/vinylimidazole polymers; condensation products of polyglycols and amines; quaternized collagen polypeptides; quaternized wheat polypeptides; polyethyleneimines; cationic silicone polymers, such as, for example, amidomethicones; copolymers of adipic acid and dimethylaminohydroxypropyldiethylenetriamine; Polyaminopolyamide and cationic chitin derivatives, such as, for example, chitosan, can be used.

The compositions according to the invention can comprise one or more of the aforementioned cationic polymers in amounts of from 0.1 to 5% by weight, preferably 0.2 to 3% by weight and particularly preferably 0.5 to 2% by weight, based on the finished compositions.

Furthermore, the compositions according to the invention can comprise film formers which, depending on the intended use, are selected from salts of phenylbenzimidazolesulfonic acid, water-soluble polyurethanes, for example C₁₀-polycarbamyl polyglyceryl ester, polyvinyl alcohol, water-soluble acrylic acid polymers/copolymers and esters or salts thereof, for example partial ester copolymers of acrylic acid/methacrylic acid, water-soluble cellulose, for example hydroxymethylcellulose, hydroxyethylcellulose, hydroxypropylcellulose, water-soluble quaterniurns, polyquaterniums, carboxyvinyl polymers, such as carbomers and salts thereof, polysaccharides, for example polydextrose and glucan, vinyl acetate/crotonate, for example available under the trade name Arlstoflex® A 60 (Clariant).

The compositions according to the invention can comprise one or more film formers in amounts of from 0.1 to 10% by weight, preferably from 0.2 to 5% by weight and particularly preferably from 0.5 to 3% by weight, based on the finished compositions.

The desired viscosity of the compositions can be established by adding further thickeners and gelling agents. Of suitability are preferably cellulose ethers and other cellulose derivatives (e.g. carboxymethylcellulose, hydroxyethylcellulose), gelatin, starch and starch derivatives, sodium alginates, fatty acid polyethylene glycol esters, agar agar, tragacanth or dextrin derivatives, in particular dextrin esters. Furthermore, metal salts of fatty acids, preferably having 12 to 22 carbon atoms, for example sodium stearate, sodium palmitate, sodium laurate, sodium arachidates, sodium behenate, potassium stearate, potassium palmitate, sodium rnyristate, aluminum monostearate, hydroxyl fatty acids, for example 12-hydroxystearic acid, 16-hydroxyhexadecanoyl acid; fatty acid amides; fatty acid alkanolamides; dibenzalsorbitol and alcohol-soluble polyamides and polyacrylamides or mixtures of such are suitable. Furthermore, crosslinked and uncrosslinked polyacrylates such as carbomers, sodium polyacrylates or polymers containing sulfonic acid, such as ammonium acryloyldimethyltaurate/VP copolymer, can be used.

Preferably, the compositions according to the invention comprise 0.01 to 20% by weight, particularly preferably 0.1 to 10% by weight, especially preferably 0.2 to 3% by weight and very particularly preferably 0.4 to 2% by weight, of thickeners and/or gelling agents, based on the finished compositions according to the invention.

Superfatting agents which can be used are preferably lanolin and lecithin, nonethoxylated and polyethoxylated or acylated lanolin derivatives and lecithin derivatives, polyol fatty acid esters, mono-, di- and triglycerides and/or fatty acid alkanolamides, where the latter simultaneously serve as foam stabilizers, which are preferably used in amounts of from 0.01 to 10% by weight, particularly preferably from 0.1 to 5% by weight and especially preferably from 0.5 to 3% by weight, based on the finished compositions according to the invention.

The antimicrobial active ingredients used are cetyltrimethylammonium chloride, cetylpyridinium chloride, benzethonium chloride, diisobutylethoxyethyldimethylbenzylammonium chloride, sodium N-laurylsarcosinate, sodium N-palmethylsarcosinate, lauroylsarcosine, N-myristoylglycine, potassium N-laurylsarcosine, trimethylammonium chloride, sodium aluminum chlorohydroxylactate, triethyl citrate, tricetylmethylammonium chloride, 2,4,4′-trichloro-2′-hydroxydiphenyl ether (triclosan), phenoxyethanol, 1,5-pentanediol, 1,6-hexanediol, 3,4,4′-trichlorocarbanilide (triclocarban), diaminoalkylamide, for example L-lysine hexadecylamide, citrate heavy metal salts, salicylates, piroctoses, in particular zinc salts, pyrithiones and heavy metal salts thereof, in particular zinc pyrithione, zinc phenol sulfate, farnesol, ketoconazole, oxiconazole, bifonazole, butoconazole, cloconazole, clotrimazole, econazole, enilconazole, fenticonazole, isoconazole, miconazole, sulconazole, tioconazole, fluconazole, itraconazole, terconazole, naftifine and terbinafine, selenium disulfide and Octopirox®, iodopropynyl butylcarbamate, methylchloroisothiazolinone, methylisothiazolinone, methyldibromoglutaronitrile, AgCl, chloroxylenol, Na salt of diethylhexyl sulfosuccinate, sodium benzoate, and phenoxyethanol, benzyl alcohol, phenoxyisopropanol, parabens, preferably butyl, ethyl, methyl and propyl paraben, and Na salts thereof, pentanediol, 1,2-octanediol, 2-bromo-2-nitropropane-1,3-diol, ethyihexyiglycerol, benzyl alcohol, sorbic acid, benzoic acid, lactic acid, imidazolidinylurea, diazolidinylurea, dimethyloldimethylhydantoin (DMDMH), Na salt of hydroxymethylglycinate, hydroxyethylglycine of Sorbic acid and combinations of these active substances.

The compositions according to the invention comprise the antimicrobial active ingredients preferably in amounts of from 0.001 to 5% by weight, particularly preferably from 0.01 to 3% by weight and especially preferably from 0.1 to 2% by weight, based on the finished compositions.

The compositions according to the invention can furthermore comprise biogenic active ingredients selected from plant extracts, such as, for example, aloe vera, and also local anesthetics, antibiotics, antiphlogistics, antiallergics, corticosteroids, sebostatics, Bisabolol®, allantoin, Phytantriol®, proteins, vitamins selected from niacin, biotin, vitamin B2, vitamin B3, vitamin B6, vitamin B3 derivatives (salts, acids, esters, amides, alcohols), vitamin C and vitamin C derivatives, (salts, acids, esters, amides, alcohols), preferably as sodium salt of the monophosphoric acid ester of ascorbic acid or as magnesium salt of the phosphoric acid ester of ascorbic acid, tocopherol and tocopherol acetate, and also vitamin E and/or derivatives thereof.

The compositions according to the invention can comprise biogenic active ingredients preferably in amounts of from 0.001 to 5% by weight, particularly preferably from 0.01 to 3% by weight and especially preferably from 0.1 to 2% by, weight, based on the finished compositions.

The compositions according to the invention can comprise astringents, preferably magnesium oxide, aluminum oxide, titanium dioxide, zirconium dioxide and zinc oxide, oxide hydrates, preferably aluminum oxide hydrate (boehmite) and hydroxides, preferably of calcium, magnesium, aluminum, titanium, zirconium or zinc, and also aluminum chlorohydrates, preferably in amounts of from 0 to 50% by weight, particularly preferably in amounts of from 0.01 to 10% by weight and especially preferably in amounts of from 0.1 to 10% by weight, based on the finished compositions according to the invention. Allantoin and bisabolol are preferred as deodorizing substances. These are preferably used in amounts of from 0.0001 to 10% by weight, based on the finished compositions according to the invention.

The compositions according to the invention can comprise microfine titanium dioxide, mica-titanium oxide, iron oxides, mica-iron oxide, zinc oxide, silicon oxides, ultramarine blue, chromium oxides as pigments/micropigments and also as sun protection filters.

The compositions according to the invention can comprise sun protection filters, preferably selected from 4-aminobenzoic acid, 3-(4′-trimethylammonium)benzylideneboran-2-one-methyl sulfate, camphorbenzalkoniummethosulfate, 3,3,5-trimethylcyclohexyl salicylate, 2-hydroxy-4-methoxybenzophenone, 2-phenylbenzimidazole-5-sulfonic acid and its potassium, sodium and triethanolamine salts, 3,3′-(1,4-phenylenedimethine)bis(7,7-dimethyl-2-oxobicyclo[2.2.1]heptane-1-methanesulfonic acid) and its salts, 1-(4-tert-butylphenyI)-3-(4-methoxyphenyl)propane-1,3-dione, 3-(4′-sulfo)benzylidenebornan-2-one and its salts, 2-ethylhexyl 2-cyano-3,3-diphenylacrylate, polymers of N-[2(and 4)-(2-oxoborn-3-ylidenemethypbenzyl]acrylamide, 2-ethylhexyl 4-methoxycinnamate, ethoxylated ethyl 4-aminobenzoate, isoamyl 4-methoxycinnamate, 2,4,6-tris[p-(2-ethylhexyloxycarbonyl)anilino]4,3,5-triaine, 2-(2H-benzotriazol-2-yl)-4-methyl-6-(2-methyl-3-(1,3,3,3-tetramethyl-1-(trimethylsilyloxy)disiloxanyl)propyl)phenol, bis(2-ethylhexyl) 4,4′-[(6-[4-((1,1-dimethylethyl)aminocarbonyl)phenylamino]-1,3,5-triazin-2,4-yl)diimino]bisbenzoate, benzophenone-3, benzophenone-4 (acid), 3-(4′-methylbenzylidene)-DL-camphor, 3-benzylidenecamphor, 2-ethylhexyl salicylate, 2-ethylhexyl 4-dimethylaminobenzoate, hydroxy-4-methoxybenzophenone-5-sulfonic acid (sulfisobenzone) and the sodium salt, 4-isopropylbenzyl salicylate, N,N,N-trimethyl-4-(2-oxoborn-3-ylidenemethyl)anilium methyl sulfate, homosalate (INN), oxybenzone (INN), 2-phenylbenzimidazole-5-sulfonic acid and its sodium, potassium and triethanolamine salts, octylmethoxycinnamic acid, isopentyl-4-methoxycinnamic acid, isoamyl-p-methoxycinnamic acid, 2,4,5-trianilino(p-carbo-2′-ethylhexyl-1′-oxy)-1,3,5-triazine (octyltriazone) phenol, 2-2(2H-benzotriazol-2-yl)-4-methyl-6-(2-methyl-3-(1,3,3,3-tetramethyl-1-(trimethylsilyl)oxy)disiloxanyppropyl (drometrizoletrisiloxane) benzoic acid, 4,4-((6-(((1,1-dimethylethyl)amino)carbonyl)phenyl)amino)-1,3,5-triazine-2,4-diyl)diimino)bis,bis(2-ethylhexyl)ester)benzoic acid, 4,4-((6-(((1,1-dimethylethyl)amino)carbonyl)phenyl)amino)-1,3,5-triazine-2,4-diyl)diimino)bis,bis(2-ethylhexyl)ester), 3-(4′-methylbenzylidene)-DL-camphor (4-methylbenzylidenecamphor), benzylidenecamphorsulfonic acid, octocrylene, polyacrylamidomethylbenzylidenecamphor, 2-ethylhexyl salicylate (octylsalicylate), ethyl-2-hexyl 4-dimethylaminobenzoate (octyldimethyl PABA), PEG-25 PABA, 2-hydroxy-4-methoxybenzophenone-5-sulfonic acid (benzophenone-5) and the Na salt, 2,2′-methylenebis-6-(2H-benzotriazol-2-yl)-4-(tetramethylbutyl)-1,1,3,3-phenol, sodium salt of 2-2′-bis(1,4-phenylene)-1H-benzimidazole-4,6-disulfonic acid, (1,3,5)-triazine-2,4-bis((4-(2-ethylhexyloxy)-2-hydroxy)phenyI)-6-(4-methoxyphenyl), 2-ethylhexyl 2-cyano-3,3-diphenyl-2-propenoate, glyceryl octanoate, di-p-methoxycinnamic acid, p-aminobenzoic acid and esters thereof, 4-tert-butyl-4′-methoxydibenzoylmethane, 4-(2-β-glucopyranoxy)propoxy-2-hydroxybenzophenone, octyl salicylate, methyl-2,5-diisopropylcinnamic acid, cinoxate, dihydroxydimethoxybenzophenone, disodium salt of 2,2′-dihydroxy-4,4′-dimethoxy-5,5′-disulfobenzophenone, dihydroxybenzophenone, 1,3,4-dimethoxyphenyl-4,4-dimethyl-1,3-pentanedione, 2-ethylhexyl dimethoxybenzylidenedioxoimidazolidinepropionate, methylenebisbenzotriazolyl tetramethylbutylphenol, phenyl dibenzimidazoletetrasulfonate, bis-ethylhexyloxyphenol methoxyphenol triazine, tetrahydroxybenzophenones, terephthalylidenedicamphorsulfonic acid, 2,4,6-tris[4,2-ethylhexyloxycarbonyl)anilino]-1,3,5-triazine, methylbis(trimethylsiloxy)silylisopentyltrimethoxycinnamic acid, amyl p-dimethylaminobenzoate, amyl p-dimethylaminobenzoate, 2-ethylhexyl p-dimethylaminobenzoate, isopropyl-p-methoxycinnamic acid/diisopropylcinnamic acid esters, 2-ethylhexyl-p-methoxycinnamic acid, 2-hydroxy-4-methoxybenzophenone, 2-hydroxy-4-methoxybenzophenone-5-sulfonic acid and the trihydrate, and also 2-hydroxy-4-methoxybenzophenone-5-sulfonate sodium salt and phenylbenzimidazolesulfonic acid.

The amount of the aforementioned sun protection filters (one or more compounds) in the compositions is preferably 0.001 to 30% by weight, particularly preferably 0.05 to 20% by weight and especially preferably 1 to 10% by weight, based on the total weight of the finished composition.

The compositions according to the invention can comprise antioxidants, preferably selected from amino acids (e.g. glycine, histidine, tyrosine, tryptophan) and derivatives thereof, imidazoles (e.g. urocanic acid) and derivatives thereof, peptides such as DL-carnosine, D-carnosine, L-carnosine and derivatives thereof (e.g. anserine), carotenoids, carotenes (e.g. α-carotene, β-carotene, lycopene) and derivatives thereof, chlorogenic acid and derivatives thereof, lipoic acid and derivatives thereof (e.g. dihydrolipoic acid), aurothioglucose, propylthiouracil and other thiols (e.g. thioredoxin, glutathione, cysteine, cystine, cystamine and the glycosyl, N-acetyl, methyl, ethyl, propyl, amyl, butyl and lauryl, palmitoyl, oleyl, γ-linoleyl, cholesteryl and glyceryl esters thereof), and also salts thereof, dilauryl thiodipropionate, distearyl thiodipropionate, thiodipropionic acid and derivatives thereof (e.g. esters, ethers, peptides, lipids, nucleotides, nucleosides and salts), and also sulfoximine compounds (e.g. buthionine sulfoximines, homocysteine sulfoximine, buthionine sulfones, penta-, hexa-, heptathionine sulfoximine) in very low tolerated doses, also (metal) chelating agents (e.g. α-hydroxyfatty acids, palmitic acid, phytic acid, lactoferrin), α-hydroxy acids (e.g. citric acid, lactic acid, malic acid), humic acid, bile acid, bile extracts, bilirubin, biliverdin, EDTA, EGTA and derivatives thereof, unsaturated fatty acids and derivatives thereof (e.g. γ-linolenic acid, linoleic acid, oleic acid), folic acid and derivatives thereof, ubiquinone and ubiquinol and derivatives thereof, vitamin C and derivatives (e.g. ascorbyl palmitate, Mg ascorbyl phosphate, ascorbyl acetate), tocopherols and derivatives (e.g. vitamin E acetate), vitamin A and derivatives (vitamin A palmitate), and coniferyl benzoate of benzoin resin, rutinic acid and derivatives thereof, α-glycosylrutin, ferulic acid, furfurylideneglucitol, carnosine, butylhydroxytoluene, butylhydroxyanisole, nordihydroguaiacic acid, nordihydroguaiaretic acid, trihydroxybutyrophenone, uric acid and derivatives thereof, mannose and derivatives thereof, zinc and derivatives thereof (e.g. ZnO, ZnSO₄), selenium and derivatives, thereof (e.g. selenomethionine), stilbenes and derivatives thereof (e.g. stilbene oxide, trans-stilbene oxide), superoxide dismutase and the derivatives suitable according to the invention (salts, esters, ethers, sugars, nucleotides, nucleosides, peptides and lipids) of these specified substances.

The antioxidants can protect the skin and the hair against oxidative stress. Preferred antioxidants here are vitamin E and derivatives thereof, and vitamin A and derivatives thereof.

The amount of the one or more antioxidants in the compositions according to the invention is preferably 0.001 to 30% by weight, particularly preferably 0.05 to 20% by weight and especially preferably 1 to 10% by weight, based on the total weight of the composition.

Furthermore, humectants selected from the sodium salt of 2-pyrrolidone-5-carboxylate (NaPCA), guanidine; glycolic acid and salts thereof, lactic acid and salts thereof, glucosamines and salts thereof, lactamide monoethanolamine, acetamide monoethanolamine, urea, hydroxy acids, panthenol and derivatives thereof, for example D-panthenol (R-2,4-dihydroxy-N-(3-hydroxypropyl)-3,3-dimethylbutamide), DL-panthenol, calcium pantothenate, panthetine, pantotheine, panthenyl ethyl ether, isopropyl palmitate, glycerol and/or sorbitol can be used, preferably in amounts of from 0.1 to 15% by weight and particularly preferably from 0.5 to 5% by weight, based on the finished compositions.

Additionally, the compositions according to the invention can comprise organic solvents. In principle, suitable organic solvents are all mono- or polyhydric alcohols. Preference is given to using alcohols having 1 to 4 carbon atoms, such as ethanol, propanol, isopropanol, n-butanol, isobutanol, t-butanol, glycerol and mixtures of said alcohols. Further preferred alcohols are polyethylene glycols with a relative molecular mass below 2000. In particular, a use of polyethylene glycol with a relative molecular mass between 200 and 600 and in amounts up to 45% by weight and of polyethylene glycol with a relative molecular mass between 400 and 600 in amounts of from 5 to 25% by weight is preferred, in each case based on the finished composition. Further suitable solvents are, for example, triacetin (glycerol triacetate) and 1-methoxy-2-propanol.

The dyes and color pigments present in the compositions according to the invention, both organic and inorganic dyes, are selected from the corresponding positive list of the Cosmetics Ordinance or the EU list of cosmetic colorants.

Also advantageous are oil-soluble natural dyes, such as, for example, paprika extracts, β-carotene and cochineal.

Pearlescent pigments are also advantageously used, e.g. pearl essence (guanine/hypoxanthine mixed crystals from fish scales) and mother-of-pearl (ground mussel shells), monocrystalline pearlescent pigments, such as, for example, bismuth oxychloride (BiOCl), layer-substrate pigments, e.g. mica/metal oxide, silver-white pearlescent pigments of TiO₂, interference pigments (TiO₂, varying layer thickness), color luster pigments (Fe₂O₃) and combination pigments (TiO₂/Fe₂O₃, TiO₂/Cr₂O₃, TiO₂/Prussian blue, TiO₂/carmine).

Within the context of the present invention, effect pigments are to be understood as meaning pigments which bring about particular optical effects as a result of their refractive properties. Effect pigments impart luster or glitter effects to the treated surface (skin, hair, mucosa) or are able to optically conceal skin unevenness and skin wrinkles through diffuse light scattering. As a particular embodiment of the effect pigments, interference pigments are preferred. Particularly suitable effect pigments are, for example, mica particles which are coated with at least one metal oxide. Besides mica, a sheet silicate, silica gel and other SiO₂ modifications are also suitable as carriers. A metal oxide which is often used for the coating is, for example, titanium oxide, to which, if desired, iron oxide can be admixed. The reflection properties can be influenced via the size and the shape (e.g. spherical, ellipsoidal, flattened, planar, nonplanar) of the pigment particles and also via the thickness of the oxide coating. Other metal oxides too, e.g. bismuth oxychloride (BiOCl), and the oxides of, for example, titanium, in particular the TiO₂ modifications anatase and rutile, and of aluminum, tantalum, niobium, zirconium and hafnium. With magnesium fluoride (MgF₂) and calcium fluoride (fluorspar, CaF₂) too, it is possible to produce effect pigments.

The effects can be controlled not only via the particle size but also via the particle size distribution of the pigment assembly. Suitable particle size distributions range, for example, from 2-50 μm, 5-25 μm, 5-40 μm, 5-60 μm, 5-95 μm, 5-100 μm, 10-60 μm, 10-100 μm, 10-125 μm, 20-100 μm, 20-150 μm, and <15 μm. A broader particle size distribution, for example of 20-150 μm, brings about glittering effects, whereas a narrower particle size distribution of <15 μm provides a uniform satin appearance.

The compositions according to the invention comprise effect pigments preferably in amounts of from 0.1 to 20% by weight, particularly preferably from 0.5 to 10% by weight and especially preferably from 1 to 5% by weight, in each case based on the total weight of the composition.

Allantoin and bisabolol are preferred as deodorizing substances. These are preferably used in amounts of from 0.0001 to 10% by weight, based on the total weight of the compositions according to the invention.

Fragrance and/or perfume oils which may be used are individual odorant compounds, e.g. the synthetic products of the ester, ether, aldehyde, ketone, alcohol and hydrocarbon types. Odorant compounds of the ester type are, for example, benzyl acetate, phenoxyethyl isobutyrate, p-tert-butylcyclohexyl acetate, linalyl acetate, dimethylbenzylcarbinyl acetate, phenylethyl acetate, linalyl benzoate, benzyl formate, ethylmethylphenyl glycinate, allyl cyclohexyl propionate, styrallyl propionate and benzyl salicylate. The ethers include, for example, benzyl ethyl ethers, the aldehydes include, for example, the linear alkanals having 8 to 18 carbon atoms, citral, citronellal, citronellyloxyacetaldehyde, cyclamenaldehyde, hydroxycitronellal, lilial and bourgeonal, the ketones include, for example, the ionones, alpha-isomethylionone and methyl cedryl ketone, the alcohols include anethol, citronellol, eugenol, geraniol, linalool, phenylethyl alcohol and terpineol, and the hydrocarbons include primarily the terpenes and balsams. Preference is given to using mixtures of different odorants which together produce a pleasing scent note.

Perfume oils can also comprise natural odorant mixtures, as are accessible from vegetable or animal sources, e.g. pine oil, citrus oil, jasmine oil, lily oil, rose oil or ylang-ylang oil, Essential oils of relatively low volatility, which in most cases are used as aromatic components, are also suitable as perfume oils, e.g. sage oil, chamomile oil, clove oil, melissa oil, mint oil, cinnamon leaf oil, linden blossom oil, juniperberry oil, vetiver oil, olibanum oil, galbanum oil and ladanurn oil.

Preferably suitable as pearlizing component are fatty acid monoalkanolamides, fatty acid dialkanolamides, monoesters or diesters of alkylene glycols, in particular ethylene glycol and/or propylene glycol or oligomers thereof, with higher fatty acids, such as, for example, palmitic acid, stearic acid and behenic acid, monoesters or polyesters of glycerol with carboxylic acids, fatty acids and metal salts thereof, ketosulfones or mixtures of the specified compounds. Particular preference is given to ethylene glycoldistearates and/or polyethylene glycoldistearates having on average 3 glycol units.

If the compositions according to the invention comprise pearlizing compounds, these are preferably present in the compositions according to the invention in an amount of from 0.1 to 15% by weight and particularly preferably in an amount of from 1 to 10% by weight, in each case based on the total weight of the composition.

The acids or alkalis used for adjusting the pH are preferably mineral acids, in particular HCl, inorganic bases, in particular NaOH or KOH, and organic acids, in particular, citric acid.

The examples and applications below are intended to illustrate the invention in more detail without, however, limiting it thereto. All of the, percentage data is percent by weight (% by wt.).

### PREPARATION EXAMPLES

### General Operating Procedure

In the preparation of the phosphoric acid esters present in the compositions according to the invention, phosphoric acid (85% strength), fatty alcohol ethoxylate and/or dial and/or polyol are used in a certain molar ratio. For this purpose, all starting materials are initially introduced in a stirred apparatus with heating mantle, separator with condenser and vacuum connection. The mixture is heated to 100° C., evacuated three times to 100 mbar and then aerated again with nitrogen. After being rendered inert for a further 4 hours (nitrogen introduction 20 liter/hour) at 100° C., the mixture is heated to 230° C. with nitrogen introduction and esterified (water discharge). The reaction times are 24 to 42 hours (calculated above 230° C. esterification temperature), in particular 40 hours. The residual acid number is then <3 mg KOH/g. This corresponds approximately to 93 to 96% conversion (based on starting acid number). When the reaction is complete, the product is cooled to 80° C. and poured into a dish, and the solidified melt is comminuted.

Phosphoric Acid Ester 1

Ester from 13.8 g of phosphoric acid, 316.1 g of polyglycol 4000 and 296.0 g of ceteareth-25 (C_{16/18} fatty alcohol+25 mol of ethylene oxide) in the molar ratio 3:2:5, residual acid number: 1.9 mg KOH/g (94% conversion), ³¹P-NMR: diester/triester=18/82 (molar ratio).

Phosphoric Acid Ester 2

Ester from 12.7 g of phosphoric acid and 701.3 g of ceteareth-50 (C₁₆₁₁₈ fatty alcohol+50 mol of ethylene oxide) in the molar ratio 1:3, residual acid number: 0.8 mg KOH/g (97% conversion), ³¹P-NMR: diester/triester=13/87 (molar ratio).

The table below shows the synergism of the phosphoric acid ester with hydrophobically modified, anionic, crosslinked polymers with regard to viscosity and development of yield point.

Phosphoric acid esters 1 and 2 were dissolved (1%) in water at 60° C., the solution was cooled to 25° C. and then admixed with 0.5% Aristoflex® HMB (ammonium acryloyldimethyltaurate/beheneth-25 methacrylate crosspolymer), and a homogeneous gel was prepared by intensive stirring. For comparison, gels/solutions of the individual components were also prepared.

The viscosity of the solutions was measured using a Brookfield DV-II rotary viscometer, rotational speed 20 s⁻¹ measured at 20° C. At <100 mPa·s, spindle 2 was used, at >10 000 mPa·s spindel 5, at >20 000 mPa·s spindle 6.

The yield points were determined using a Bohlin rheometer using the Casson method (plot shear rate^{1/2} vs. shear stress^{1/2}) in the 0-1 s⁻¹ shear rate region at 25° C.

TABLE 1
Viscosity behavior and formation of yield points

| | Anionic, crosslinked, | | | | |
|---|---|---|---|---|---|
| | hydrophobically | | | | |
| Phosphoric acid ester | modified polymer | Water | | Viscosity | Yield point |
| [% by wt.] | [% by wt.] | [% by wt.] | Clarity | [mPa · s] | [Pa] |
| 0 | Ammonium | 99.5 | clear | 11 000 | 8.4 |
| | acryloyldimethyltaurate/ | | | | |
| | beheneth-25 meth-acrylate | | | | |
| | crosspolymer | | | | |
| | [0.5% by wt.] | | | | |
| Phosphoric acid ester 1 | Ammonium | 98.5 | clear | 32 800 | 35.0 |
| [1.0% by weight] | acryloyldimethyltaurate/ | | | | |
| | beheneth-25 meth-acrylate | | | | |
| | crosspolymer | | | | |
| | [0.5% by wt.] | | | | |
| Phosphoric acid ester 2 | Ammonium | 98.5 | clear | 28 000 | 26.6 |
| [1.0% by weight] | acryloyldimethyltaurate/ | | | | |
| | beheneth-25 meth-acrylate | | | | |
| | crosspolymer | | | | |
| | [0.5% by weight] | | | | |
| Phosphoric acid ester 1 | 0 | 99.0 | clear | 60 | 0 |
| [1.0% by weight] | | | | | |
| Phosphoric acid ester 2 | 0 | 99.0 | clear | 50 | 0 |
| [1.0% by weight] | | | | | |

It can be seen from the results listed in table 1 that the combination according to the invention of the phosphoric acid esters and the anionic, crosslinked, hydrophobically modified polymers brings about both a significant increase in the yield point of the gel, and also considerably increases the viscosity under the measurement conditions chosen. Moreover, the gels comprising phosphoric acid ester and anionic, crosslinked, hydrophobically modified polymer are clear and, compared with a gel based only on the polysulfonic acid, offer significantly improved pick-up, i.e. the gels can be taken up easily on the finger without the gel liquefying and sliding off.

### FORMULATION EXAMPLES

### Formulation Example 1

### Vitamin C Gel

| | | |
|---|---|---|
| A | Phosphoric acid ester 2 | 1.0% |
| | Genapol ® T 250 (Clariant) | 1.5% |
| | Ceteareth-25 | |
| B | Water | ad 100% |
| C | Ascorbic acid | 3.0% |
| D | Aristoflex ® HMB (Clariant) | 1.2% |
| | Ammonium acryloyldimethyltaurate/beheneth-25 | |
| | methacrylate crosspolymer | |

Preparation:

I Dissolve A in B with stirring and heat to ca. 50° C.

II Add C to I.

Ill Add D and stir until a homogeneous gel has formed.

### Formulation Example 2

### Facial Anti-Ageing Gel

| | | |
|---|---|---|
| A | Genapol ® T 250 (Clariant) | 1.5% |
| | Ceteareth-25 | |
| | Phosphoric acid ester 2 (Clariant) | 1.0% |
| B | Water | ad 100% |
| C | Aristoflex ® HMB (Clariant) | 1.6% |
| | Ammonium acryloyldimethyltaurate/beheneth-25 | |
| | methacrylate crosspolymer | |
| D | Glycol acid 30% * | 6.0% |
| | Preservative | q.s. |

* Adjusted to pH 4 with NaOH (content based on free glycol acid)

Preparation:

I Dissolve A in B with stirring and gentle heating.

II Add C to I and stir until the gel is free from lumps (ca. 300 rpm using a finger stirrer).

III Add the contents of D to II and stir until the formulation is homogeneous.

### Formulation Example 3

### Facial Anti-Ageing Cream Gel

| | | |
|---|---|---|
| A | Phosphoric acid ester 1 | 1.0% |
| B | Water | ad 100% |
| C | Mineral oil | 5.0% |
| | SilCare ® Silicone 31M50 (Clariant) | 3.0% |
| | Caprylyl trimethicone | |
| D | Aristoflex ® HMB (Clariant) | 1.5% |
| | Ammonium acryloyldimethyltaurate/beheneth-25 | |
| | methacrylate crosspolymer | |
| E | Glycol acid 30% * | 6.0% |
| | Preservative | q.s. |
| F | Genapol ® LA 070 (Clariant) | 3.0% |
| | Laureth-7 | |

* Adjusted to pH 4 with NaOH (content based on free glycol acid)

Preparation:

I Dissolve A in B with stirring and gentle heating.

II Add D to C.

III Add I to II and stir until the gel is free from lumps (ca. 300 rpm using a finger stirrer).

IV Add the components of E to III.

V Add F to IV and stir until the formulation is homogeneous.

VI Finally homogenize the cream gel.

### Formulation Example 4

### O/W Self-Tanning Cream

| | | |
|---|---|---|
| A | Hostaphat ® CC 100 (Clariant) | 1.0% |
| | Cetyl phosphate | |
| | Glyceryl stearate | 0.5% |
| | Cetearyl alcohol | 0.5% |
| | Paraffin oil, n.v. | 8.0% |
| | Isopropyl palmitate | 7.0% |
| | SilCare ® Silicone 41M15 (Clariant) | 1.0% |
| | Caprylyl methicone | |
| B | Aristoflex ® HMB (Clariant) | 0.7% |
| | Ammonium acryloyldimethyltaurate/beheneth-25 | |
| | methacrylate crosspolymer | |
| C | Phosphoric acid ester 2 | 1.0% |
| D | Water | ad 100% |
| | Hostapon ® CLG (Clariant) | 0.5% |
| | Sodium lauroyl glutamate | |
| | Glycerol | 5.0% |
| E | Tocopheryl acetate | 1.0% |
| | Perfume | 0.2% |
| | Preservative | q.s. |
| F | Dihydroxyacetone | 5.0% |
| | Water | 8.0% |
| G | Sodium hydroxide solution | q.s. |

Preparation:

I Melt A at 80° C.

II Stir B into A.

III Dissolve C in D with stirring and heating to about 50° C., then add to II.

IV At 30° C., stir E into III.

V Dissolve dihydroxyacetone in the water and, add F to IV.

VI Adjust pH to ca. 4 with G, if necessary.

### Formulation Example 5

### Sun Protection Cream Gel

| | | |
|---|---|---|
| A | Eusolex ® 232 | 8.0% |
| | Phenylbenzimidazole sulfonic acid | |
| B | Water | ad 100% |
| C | Phosphoric acid ester 2 | 1.0% |
| D | Aristoflex ® HMB (Clariant) | 1.0% |
| | Ammonium acryloyldimethyltaurate/beheneth-25 | |
| | methacrylate crosspolymer | |
| E | Tegosoft TN | 5.0% |
| | C₁₂₋₁₅ alkyl benzoate | |
| | SilCare ® Silicone 15M50 (Clariant) | 4.0% |
| | Phenyl trimethicone | |
| | Eusolex ® 9020 | 3.0% |
| | Methoxydibenzoylmethane | |
| | Velsan ® D8P-3 (Clariant) | 3.5% |
| | Isopropyl PPG-2 isodeceth-7 carboxylate | |
| F | Nipaguard ® MPA (Clariant) | q.s. |
| | Benzyl alcohol (and) methylparaben | |
| | (and) propylparaben | |
| | Genapol ® LA 070 (Clariant) | 1.5% |
| | Laureth-7 | |
| | SilCare ® Silicone SEA (Clariant) | 0.5% |
| | Trideceth-9 PG-amodimethicone and trideceth-12 | |
| | Perfume | q.s. |

Preparation:

I Mix A and B and neutralize (adjust pH to ca. 7.3).

II Dissolve C in I with stirring and gentle heating.

III Add D and stir until a homogeneous gel has formed.

IV Mix the components of E, dissolve with gentle heating and then add to III.

V Finally, add F to VI.

### Formulation Example 6

### Pigment Shower Bath

| | | |
|---|---|---|
| A | Water | ad 100% |
| B | Phosphoric acid ester 1 | 2.0% |
| C | Aristoflex ® HMB (Clariant) | 0.7% |
| | Ammonium acryloyldimethyltaurate/beheneth-25 | |
| | methacrylate crosspolymer | |
| D | Genapol ® LRO liquid (Clariant) | 30.0% |
| | Sodium laureth sulfate | |
| | Perfume | 0.5% |
| | Preservative | q.s. |
| E | Hostapon ® CGN (Clariant) | 4.0% |
| | Sodium cocoyl glutamate | |
| F | Cirebelle 104 | 0.2% |
| | Synthetic wax | |

Preparation:

I Dissolve B in A with stirring and heating to ca. 50° C.

II Add C to I and stir until an almost homogeneous gel has formed.

III Mix the components of D and add to II With stirring, further stir until the formulation is homogeneous.

IV Add E to III.

V Add F to IV.

VI Finally, if necessary, adjust the pH.

### Formulation Example 7

### Facial Toner

| | | |
|---|---|---|
| A | Glycerol | 10.0% |
| | Polyglycol 400 (Clariant) | 5.0% |
| | PEG-8 | |
| | Panthenol | 0.5% |
| | Perfume | 0.2% |
| | Preservative | q.s. |
| | Allantoin (Clariant) | 0.1% |
| | Niacinamide | 0.1% |
| | Extrapon Hamamelis | 1.0% |
| | Water, witch hazel distillate, | |
| | SD alcohol 39-C, butylene glycol | |
| B | Water | ad 100% |
| C | Phosphoric acid ester 1 | 0.5% |
| D | Aristoflex ® HMB (Clariant) | 0.2% |
| | Ammonium acryloyldimethyltaurate/beheneth-25 | |
| | methacrylate crosspolymer | |

Preparation:

I Dissolution of C in B at ca. 70° C.

II Dissolution of D with stirring in I.

Ill Addition of the components of A to II.

### Formulation Example 8

### Whitening Gel

| | | |
|---|---|---|
| A | Water | ad 100% |
| | Arginine | 1.10% |
| B | Phosphoric acid ester 2 (Clariant) | 2.00% |
| C | Dipropylene glycol | 8.00% |
| | Genapol ® C 100 (Clariant) | 0.60% |
| | Coceth-10 | |
| | Sodium citrate* 2H₂O | 0.09% |
| | Citric acid 10.0% | 0.10% |
| | Nipagin ® M (Clariant) | 0.20% |
| | Methylparaben | |
| | Ascorbic acid 2-glucoside | 2.00% |
| D | Aristoflex ® HMB (Clariant) | 1.50% |
| | Ammonium acryloyldimethyltaurate/beheneth-25 | |
| | methacrylate crosspolymer | |

Preparation:

I Dissolution of the components of A.

II Dissolution of B in 1 at ca. 70° C.

Ill Dissolution of C with stirring in II.

IV Dissolution of D in III.

### Formulation Example 9

### Make-Up Remover

| | | |
|---|---|---|
| A | Velsan ® P8-3 (Clariant) | 5.0% |
| | Isopropyl C₁₂₋₁₅ pareth-9 carboxylate | |
| B | Water | ad 100% |
| C | Phosphoric acid ester 2 (Clariant) | 1.0% |
| D | Hostapon ® CGN (Clariant) | 2.0% |
| | Sodium cocoyl glutamate | |
| | Genagen ® CAB (Clariant) | 3.0% |
| | Cocamidopropylbetaine | |
| | Allantoin (Clariant) | 0.3% |
| | Aristoflex ® PEA (Clariant) | 1.0% |
| | Polypropylene terephthalate | |
| | 1,6-Hexanediol | 2.0% |
| | 1,2-Propanediol | 2.0% |
| | Polyglycol 400 (Clariant) | 2.0% |
| | PEG-8 | |
| | Panthenol | 0.5% |
| | Lutrol F 127 | 3.0% |
| | Poloxamer 407 | |
| | Preservative | q.s. |
| E | Aristoflex ® HMB (Clariant) | 0.7% |
| | Ammonium acryloyldimethyltaurate/beheneth-25 | |
| | methacrylate crosspolymer | |
| F | Genapol ® LA 070 (Clariant) | 2.0% |
| | Laureth-7 | |

Preparation:

I Dissolution of C in B at ca. 70° C.

II Dissolution of E with stirring in I.

III Dissolution of the components of D in II.

IV Dissolution of A in III.

V Dissolution of F in IV.

### Formulation Example 10

### Deodorant Gel

| | | |
|---|---|---|
| A | Octopirox ® (Clariant) | 0.1% |
| | Piroctone olamine | |
| B | Emuisogen ® HCP 049 (Clariant) | 10.0% |
| | PEG-40 hydrogenated castor oil and propylene glycol | |
| | Perfume | 0.2% |
| C | Water | ad 100% |
| D | Phosphoric acid ester 2 (Clariant) | 3.0% |
| | Aristoflex ® HMB (Clariant) | 0.2% |
| | Ammonium acryloyldimethyltaurate/beheneth-25 | |
| | methacrylate crosspolymer | |
| E | Citric acid | q.s. |

Preparation:

I Dissolve A in B.

II Dissolve D in C with stirring and gentle heating, then add II to I.

III If necessary, adjust the pH to 6.0 with E.

### Formulation Example 11

### Hydrogen Peroxide Gel

| | | |
|---|---|---|
| A | Phosphoric acid ester 1 | 1.50% |
| | Aristoflex ® HMB | 1.00% |
| | Ammonium acryloyldimethyltaurate/beheneth-25 | |
| | methacrylate crosspolymer | |
| | Genapol ® T 250 (Clariant) | 2.00% |
| | Ceteareth-25 | |
| B | Water | ad 100% |
| C | Phosphoric acid | 0.04% |
| | Sodium dihydrogenphosphate | 1.00% |
| D | Hydrogen peroxide 30% strength | 18.00% |

Preparation:

I Dissolve A in B with stirring and heating to 50° C.

II Add C at 25° C.

III Add D at room temperature.

1. A composition comprising:
I) at least one anionic, crosslinked, hydrophobically modified polymer, obtained by copolymerization of
a) at least one monomer of the formula (IV) 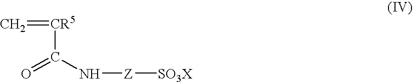
wherein R⁵ is hydrogen, methyl or ethyl, Z is (C₁-C₈)-alkylene and X is hydrogen, lithium, sodium, potassium, magnesium, calcium, ammonium, monoalkylammonium, dialkylammonium, trialkylammonium or tetraalkylammonium, where the alkyl substituents of the ammonium ions, independently of one another, are (C₁-C₂₂)-alkyl radicals or (C₂-C₁₀)-hydroxyalkyl radicals,
b) at least one monomer of the formula (V)
R²—Y—(R³—O)ₓ—R⁴. (V)
wherein R² is a vinyl, allyl, acryloyl or methacryloyl radical, R³ is (C₂-C₄)-alkylene, R⁴ is a linear or branched, saturated alkyl group having 6 to 50, carbon atoms, x is an integer from 0 to 500, and Y is O, S or NH, and
c) at least one crosslinker,
d) and optionally at least one monomer and
II) at least one phosphoric acid ester
where the at least one phosphoric acid ester comprises:
1) at least one structural unit derived from substances of component a), where the substances of component a) are selected from the group consisting of orthophosphoric acid and at least one derivative of orthophosphoric acid.
2) at least one structural unit derived from substances of component b), where the substances of component b) are selected from at least one compound of the formula (I)
R¹—O—(CH₂CH₂O)ᵤ(C₃H₆O)ᵥ(DO)_{w}—H (I)
wherein
R¹ is a linear or branched, saturated alkyl group having 6 to 30, carbon atoms, is a linear or branched, mono- or polyunsaturated alkenyl group having 6 to 30, carbon atoms or is an aryl group,
D is a linear or branched saturated alkylene group having 4 to 20 carbon atoms, is a linear or branched mono- or polyunsaturated alkenylene group having 4 to 20 carbon atoms or is —CH(aryl)CH₂—,
u is a number from 0 to 200,
v is a number from 0 to 100,
w is a number from 0 to 100, and
where the groups CH₂CH₂O, C₃H₆O and DO from the compounds of formula (I) may be arranged blockwise or in random distribution, and the sum u+v+w is ≧20,
3) optionally at least one structural unit derived from substances of component c), where the substances of component c) are selected from one or more diols of the formula (II)
HO—(CH₂CH₂O)ₐ(C₃H₆O)_{b}(DO)_{c}—H (II)
wherein
D is defined as in formula (I),
a is a number from 0 to 800,
b is a number from 0 to 100,
c is a number from 0 to 100,
where the sum a+b+c is ≧1, and the groups CH₂CH₂O, C₃H₆O and DO from the compounds of the formula (II) may be arranged blockwise or in random distribution, and
4) optionally at least one structural unit derived from a polyol having more than 2 OH groups.

2. A composition as claimed in claim 1, which comprises, in component I), at least one anionic, crosslinked, hydrophobically modified polymer obtained by copolymerization of
a) acrylic acid, methacrylic acid or C₁-C₄ alkyl esters of acrylic acid or of methacrylic acid,
b) at least one monomer of the formula (III)
R²—Y—(R³—O)ₓ—R⁴ (III)
wherein R² is a vinyl, allyl, acryloyl or methacryloyl radical, R³ is (C₂-C₄)-alkylene, R⁴ is a linear or branched, saturated alkyl group having 6 to 50, carbon atoms, x is an integer from 0 to 500, and Y is O, S or NH, and
c) at least one crosslinker,
d) and optionally at least further monomer.

3. A composition as claimed in claim 1, wherein, in the at least one monomer of the formula (IV), R⁵ is hydrogen, Z is —C(CH₃)₂—CH₂— and X is defined in claim 1, and in the at least one monomer of the formula (V), R² is methacryloyl, R³ is CH₂CH₂, R⁴ is a radical selected from the group consisting of stearyl, lauryl, cocoyl, undecyl, behenyl, cetearyl, cetyl and myristyl, x is an integer from 3 to 50, and Y is O.

4. The composition as claimed in claim 1, wherein the at least one phosphoric acid ester in component II), is at least one phosphoric acid trimester of the formula (III) 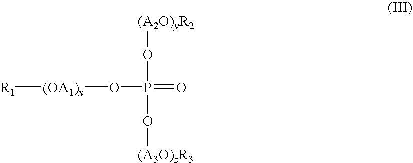
wherein
R₁, R₂ and R₃ may be identical or different and are a linear or branched, saturated alkyl group having 6 to 30, carbon atoms, are a linear or branched, mono- or polyunsaturated alkenyl group having 6 to 30, carbon atoms or are an aryl group, which may be substituted by 1 to 3 branched alkyl groups, which, in each case independently of one another, comprise 3 to 18 and preferably 4 to 12, carbon atoms,
the individual groups (OA₁)ₓ, (A₂O)_{y} and (A₃O)_{z}, in each case independently of one another, consist of units selected from the group consisting of CH₂CH₂O, C₃H₆O and C₄H₈O and where the units CH₂CH₂O, C₃H₆O and C₄H₈O may be arranged within the individual groups (OA₁)ₓ, (A₂O)_{y} and (A₃O)_{z} blockwise or in random distribution, and
x, y and z, in each case independently of one another, are a number from 25 to 150.

5. A composition as claimed in claim 1, wherein the at least one phosphoric acid ester in component II), is at least one phosphoric acid trimester which has been obtained by reacting phosphoric acid or a phosphoric acid derivative, with a fatty alcohol ethoxylate, and where the fatty alcohol radicals are derived from alcohols selected from the group consisting of octanol, decanol, dodecanol, tetradecanol, hexadecanol, octadecanol, eicosanol, behenyl alcohol, fatty alcohols with C chain cuts between 8 and 22, and monounsaturated fatty alcohols.

6. A composition as claimed in claim 1, wherein the at least one phosphoric acid ester in component II), is at least one phosphoric acid trimester which has been obtained by reacting phosphoric acid or a phosphoric acid derivative, C_{16/18}-fatty alcohol ethoxylates having 25 to 150 ethylene oxide units.

7. A composition as claimed in claim 1, wherein the at least one phosphoric acid ester in component II), is at least one phosphoric acid ester which comprise structural units derived from diols of the formula (II).

8. A composition as claimed in claim 7, wherein the at least one phosphoric acid ester in component II), is at least one phosphoric acid ester which comprise structural units derived from diols selected from the group consisting of ethylene glycol, diethylene glycol, triethylene glycol, tetraethylene glycol, polyethylene glycol (PEG) with molecular weights of from 200 to 35 000, propylene glycol, dipropylene glycol, tripropylene glycol, polypropylene glycol, polybutylene glycol, copolymers of ethylene oxide and propylene oxide with molecular weights of from 200 to 35 000, 1,2-butanediol, 1,3-butanediol, 1,4-butanediol, 1,2-pentanediol, 1,3-pentanediol, 1,4-pentanediol, 1,5-pentanediol, 1,2-hexanediol, 1,3-hexanediol, 1,4-hexanediol, 1,5-hexanediol, 1,6-hexanediol and 1,12-dodecanediol.

9. A composition as claimed in claim 1, which comprises the at least one anionic, crosslinked, hydrophobically modified polymer of component I), based on the total weight of the composition, in an amount of from 0.1 to 3.0% by weight.

10. A composition as claimed in claim 1, which comprises the at least one phosphoric acid ester of component II), based on the total weight of the composition, in an amount of from 0.1 to 5.0% by weight.

11. A composition as claimed in claim 1, which is a cosmetic, pharmaceutical or dermatological composition.

12. A composition as claimed in claim 1, which is in the form of a gel.

13. A composition as claimed in claim 1, further comprising hydrogen peroxide or at least one hydrogen peroxide-releasing substance.

14. A composition as claimed in claim 1, wherein R¹ is a phenyl group which may be substituted by 1 to 3 branched alkyl groups, which in each case independently of one another comprise 3 to 18 carbon atoms.

15. A composition as claimed in claim 1, wherein the at least one phosphoric acid ester in component II), is at least one phosphoric acid trimester which have been obtained by reacting phosphoric acid or a phosphoric acid derivative, with C_{16/18}-fatty alcohol ethoxylate having 25 ethylene oxide units, C_{16/18}-fatty alcohol ethoxylate having 50 ethylene oxide units or C_{16/18}-fatty alcohol ethoxylate having 80 ethylene oxide units.
